(19) United States
(12) Patent Application Publication
(10) Pub. No.: US 2024/0336758 A1
(43) Pub. Date: Oct. 10, 2024

(54) METHOD FOR MANUFACTURING FOAMED MICROCELLULAR SILICONE RESIN CURED PRODUCT USING SUPERCRITICAL FLUID AND FOAMED MICROCELLULAR SILICONE RESIN CURED PRODUCT MANUFACTURED THEREBY

(71) Applicant: MEGEN LIMITED, Seoul (KR)

(72) Inventors: Ki Young YOUN, Seoul (KR); Jae Koo KIM, Daejeon (KR); Sung Don HONG, Seoul (KR)

(21) Appl. No.: 18/626,326

(22) Filed: Apr. 4, 2024

(30) Foreign Application Priority Data
Apr. 7, 2023 (KR) 10-2023-0046292

(51) Int. Cl. C08J 9/12; A61L 27/18; A61L 27/56

(52) U.S. Cl. CPC C08J 9/122; A61L 27/18; A61L 27/56; C08J 2203/06; C08J 2203/08; C08J 2205/044; C08J 2205/052; C08J 2383/04

(57) ABSTRACT

A method for manufacturing a foamed microcellular silicone resin cured product which has uniform foaming and excellent dimensional accuracy, using a supercritical fluid, is described. A foamed microcellular silicone resin cured product manufactured thereby is described.

### CROSS-REFERENCE TO RELATED APPLICATIONS

This application claims priority under 35 U.S.C. § 119 to Korean Patent Application No. 10-2023-0046292, filed on Apr. 7, 2023 in the Korean Intellectual Property Office, the disclosure of which is incorporated herein by reference in its entirety.

### TECHNICAL FIELD

The present disclosure relates to a method for manufacturing a foamed microcellular silicone resin cured product using a supercritical fluid and a foamed microcellular silicone resin cured product manufactured thereby, and more particularly, to a method for manufacturing a foamed microcellular silicone resin cured product which may implement a silicone foamed molded product having uniform foaming and excellent dimensional accuracy and having a foamed cell structure of 1 to 10 μm, and a foamed microcellular silicone resin cured product manufactured thereby.

### BACKGROUND

Foam molding is a method for manufacturing a product by producing bubbles and uniformly dispersing the bubbles in a polymer resin. Since a large proportion of a foamed molded product is occupied by bubbles, material costs may be significantly reduced, weights may be reduced, and insulation or elasticity due to bubbles may be obtained.

In addition, conventionally, an engineering method including manufacturing a foam preform followed by a foaming process by gas filling and rapid depressurization in a foaming container, and a supercritical foaming mucell molding engineering method for a process technology of making a plastic in a fine foaming state by mixing carbon dioxide or nitrogen in a supercritical state with a resin are known. The supercritical foaming mucell molding engineering method is a technology which may reduce costs of injection molded articles in a large range by micronization (5-50 μm) of foamed cells.

The conventional manufacturing methods are suitable for industrial and office use and most of them may be appropriate for weight reduction, cost saving, sound absorption, insulation, shock absorption, and the like, however, since the entire process is not performed inside one high-temperature and high-pressure reactor, it is not easy to precisely control temperature and pressure, for example, carbon dioxide or nitrogen in a supercritical fluid state leaves supercritical fluid formation conditions during the injection of carbon dioxide or nitrogen into a plastic (or resin) and is converted into a simple gas phase rather than a supercritical state, and thus, there is a limitation in preventing formation of non-uniform foamed cell morphology throughout the product.

Accordingly, there is a limitation in implementing a foamed microcellular silicone resin cured product having biocompatibility, biofunctionality, uniform foaming, and excellent dimensional accuracy so that the product may be applied to a human body insertion type implant. In particular, since it is used inside a human body, there is a limitation in implementing uniform and fine foamed cells having a maximum size of 10 μm or less which are permeable to oxygen but impermeable to body fluids and moisture.

### RELATED ART DOCUMENTS

### Patent Document

(Patent Document 1) Korean Patent Registration No. 10-2376525 (International Application No. PCT/CN2018/111452)
(Patent Document 2) Chinese Patent No. 115466515 A (Dec. 13, 2022)
(Patent Document 3) Japanese Patent No. 5681347 B2 (Publication No. 2010-162765 A)

### SUMMARY

An embodiment of the present invention is directed to providing a method for manufacturing a foamed microcellular silicone resin cured product, of which the entire process is performed inside one huge container (furnace) which allows a supercritical fluid state to be maintained consistently and continuously until a foaming process.

Another embodiment of the present invention is directed to providing a method for manufacturing a foamed microcellular silicone resin cured product which has uniform forming and excellent dimensional accuracy and has a foamed cell structure of 1 to 10 μm by control using variables such as time, temperature, and pressure under a supercritical fluid, using inert harmless gas such as carbon dioxide as a supercritical fluid, and a foamed microcellular silicone resin cured product manufactured thereby.

Still another embodiment of the present invention is directed to providing a foamed microcellular silicone resin cured product which is appropriate for biocompatibility with a living body and biofunctionality to be applied to a human body insertion type implant.

In one general aspect, a method for manufacturing a foamed microcellular silicone resin cured product using a supercritical fluid, includes: (a) adding a thermocurable silicone resin to a container (furnace) and injecting carbon dioxide; (b) heating and pressurizing the carbon dioxide in the container to form a supercritical fluid state; (c) further performing heating and pressurization in the supercritical fluid state to form foamed microcells; and (d) performing heating to a temperature at which the thermocurable silicone resin is cured while pressurizing.

In an exemplary embodiment, step (c) may include: at or below the curing temperature of step (d), (c1) further performing first heating and pressurization in the supercritical fluid state to form a foaming nucleus; and (c2) further performing second heating and pressurization to or above the temperature and the pressure of the step (c1) to form a foamed microcellular structure.

In an exemplary embodiment, after the step (d), (e) performing depressurization while maintaining a temperature at or above the curing temperature of step (d) for a certain period of time to stabilize the foamed microcellular structure may be further included.

In an exemplary embodiment, a temperature condition for forming foamed microcells in step (c) may be 31 to 60° C. and a pressure condition therefor may be 74 to 350 bar.

In an exemplary embodiment, a temperature condition for forming a foaming nucleus in step (c1) may be 31 to 45° C. and a pressure condition therefor may be 74 to 250 bar; and a temperature condition for forming the foamed microcellular structure in step (c2) may be 45 to 60° C. and a pressure condition therefor may be 250 to 350 bar.

In an exemplary embodiment, the stabilizing of the foamed microcellular structure in step (e) may be performed by depressurizing to 1 bar while maintaining the temperature at 85 to 105° C.

In an exemplary embodiment, a pressure reducing rate of the step (d) may be 0.1 to 10 bar/s at or above the pressure of the supercritical fluid state condition of the step (b).

In an exemplary embodiment, the pressure reducing rate of the step (d) may be 0.01 to 0.1 bar/s from the pressure of the supercritical fluid state condition or lower of the step (b) to 1 bar.

In an exemplary embodiment, a stabilization pressure reducing rate of the step (e) may be 0.001 to 0.01 bar/s at a pressure of 1 bar or less.

In an exemplary embodiment, the step (c) may be performed for 20 minutes to 12 hours.

In an exemplary embodiment, the step (c1) may be performed for 10 minutes to 2 hours; and
the step (c2) may be performed for 10 minutes to 10 hours.

In an exemplary embodiment, the step (e) may be performed for 3 hours to 4 hours.

In an exemplary embodiment, the thermocurable silicone resin may be cured at 80 to 150° C.

In an exemplary embodiment, the carbon dioxide of step (a) may have a purity of 99.9 to 99.999%.

In another general aspect, a foamed microcellular silicone resin cured product manufactured by any one of the above methods is provided.

In an exemplary embodiment, the cured product may have a closed cell structure of a round, oval, or irregular shape of 1 to 10 μm.

In an exemplary embodiment, the cured product may be used as an implant which is inserted or attached to one or more selected from the group consisting of a chest, breasts, buttocks, a nose, a chin, a forehead, calves, thighs, and wrinkles of a human body.

Other features and aspects will be apparent from the following detailed description, the drawings, and the claims.

### BRIEF DESCRIPTION OF THE DRAWINGS

Hereinafter, the drawings described later are only some exemplary embodiments of the present invention, and a person with ordinary skill in the art to which the present invention pertains may obtain another drawing based on the drawings without creative labor.

FIG. 1 is a cross-sectional view showing integrated batch foaming equipment based on using supercritical fluid carbon dioxide according to an exemplary embodiment of the present invention.

FIG. 2 shows implants manufactured according to (a) Example 1, (b) Example 2, and (c) Example 3, as foamed microcellular silicone resin cured products manufactured using the integrated batch foaming equipment.

FIG. 3 is photographs showing appearance differences depending on (a) a nitrogen purity of 99.9%, (b) a carbon dioxide purity of 99.9%, (c) a carbon dioxide purity of 99.99%, and (d) a carbon dioxide purity of 99.999%, as foamed microcellular silicone resin cured products manufactured according to an exemplary embodiment of the present invention.

FIG. 4 is SEM photographs of foamed microcellular silicone resin cured products at a carbon dioxide purity of (a) 99.9%, (b) 99.99%, and (c) 99.999% when a saturation time was 30 minutes, and SEM photographs of foamed microcellular silicone resin cured products manufactured at a carbon dioxide purity of (d) 99.9%, (e) 99.99%, and (f) 99.999% when a saturation time was 5 hours, as foamed microcellular silicone resin cured products manufactured according to an exemplary embodiment of the present invention.

### DETAILED DESCRIPTION OF MAIN ELEMENTS

10: container
10a: gas inlet
10b: gas outlet
11: mold
12: basket

### DETAILED DESCRIPTION OF EMBODIMENTS

Hereinafter, the preferred exemplary embodiments and the physical properties of each component of the present invention will be described in detail, but these are for describing the present invention in detail so that a person with ordinary skill in the art to which the present invention pertains may easily carry out the invention and do not mean that the technical idea and the scope of the present invention are not limited thereby.

A conventional foam molding technology needs a process of mixing a polymer melt which is a high-viscosity fluid and a gas foaming agent at a low viscosity using extrusion and injection engineering methods and then retaining the mixture for a certain period of time in a state where optimal temperature and pressure conditions are satisfied to stabilize the mixture in a state of a uniform molten mixture. However, when the molten mixture is retained for a certain period of time before being discharged to the outside, pressure loss by a pressure difference occurring in an interface is caused even when a certain pressure is maintained, and thus, a part of a foaming agent is released and does not prevent premature foaming, thereby forming a molten mixture in a partially foamed state. When the mixture is extruded or injected and then filled into a cavity of a mold, irregularities occur on an outer surface of the foam molded product, so that the surface becomes rough and overall mechanical properties become inferior. In particular, when injection is performed using a partially foamed molten mixture, a part of gas which has been already foamed before an injection process leaks to the outside and it becomes difficult to manufacture a molded product having uniform foamed cell morphology. In addition, when the partially foamed molten mixture is filled, the molten mixture is filled in a state in which the pressure is higher than necessary, and thus, formed overall non-uniform foamed cell morphology becomes non-uniform and related mechanical and chemical properties become poor. In addition, since the partially foamed molten mixture has a non-uniform foaming state, it is difficult to continuously maintain a stable production process. For the reason described above, the conventional foam molding device is known to have a high defect rate, and is suitable for industrial and office products rather than high-quality medical equipment. Since manufacture of a foamed preform by a multi-step extrusion and injection process, which was devised for solving the above problems, followed by a foaming process by gas filling and rapid depressurization in the foaming container also has no fundamental difference from the above process, similar problems are exposed.

In addition, a conventional foamed cell manufacturing technology adopts a method of implementing a foamed cellular structure by injecting a gaseous phase into a thermoplastic material itself or a foamed preform and then releasing pressure at a rapid rate. Since gas trapped in a polymer structure of a thermoplastic plastic material or a foamed preform rapidly escapes to the outside by the method, several individual foamed cells clump together to form one large open foamed cell structure rather than an individual closed foamed cell. To describe in more detail, individual closed foamed microcells may be formed on a central part of a finished product, but it may be often observed that the individual foamed cells clump together toward the outside to form huge open foamed cells. In this case, even in the case of using a supercritical ultra-high purity fluid, when pressure release conditions proceed rapidly, a foamed cell structure which may only grow from the center to the outside of a final product may be obtained.

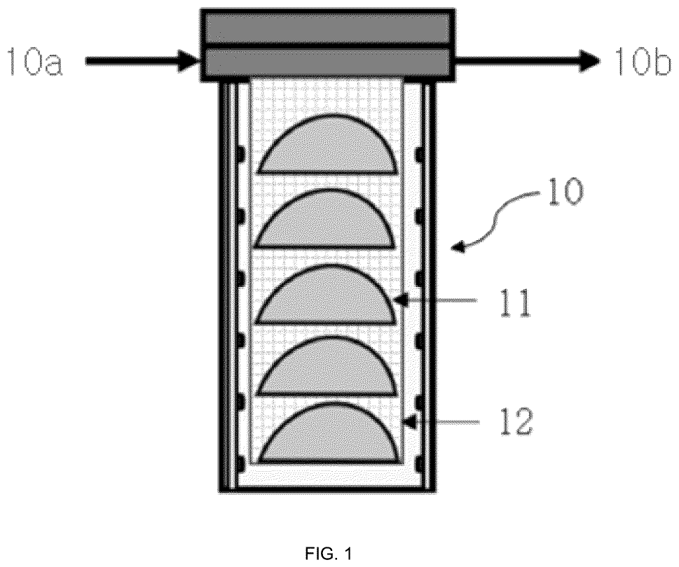

In the present invention, in a state in which a thermocurable silicone resin is not cured, a supercritical fluid state is formed by gas injection even under temperature and pressure conditions where ultra-high-purity carbon dioxide becomes a supercritical fluid, a foaming nucleus is formed by pressurization, and a final foamed microcellular structure is formed by expansion through formation and diffusion of foamed microcells based on the foaming nucleus through additional pressurization (injecting ultra-high-purity carbon dioxide) and saturation with supercritical carbon dioxide for a certain period of time. In an exemplary embodiment used in the present invention, integrated batch foaming equipment is as shown in FIG. 1. The present invention will be described in more detail, as follows.

As a first step, a thermocurable silicone resin block is kneaded into a uniform mixture by kneader equipment and then added in a certain amount to a product-shaped mold, 1 to 12 molds are laminated inside a basket and disposed under the exact center of a high-temperature and high-pressure container (furnace), and then carbon dioxide is injected to a certain temperature and a certain pressure.

As a second step, the carbon dioxide in the container is heated and pressurized to form a supercritical fluid state.

The supercritical fluid is in a state of being uniformly mixed without a mutual boundary between a liquid phase and a gaseous phase under conditions at or above certain temperature and pressure, and its overall physical and rheological behaviors seem to be similar to those of pure gas, but the effects on foamed cell morphology are significantly different. Further, though normal solid, liquid, and gas involve a change in state depending on temperature and pressure, the supercritical fluid does not change to a liquid or gaseous state under supercritical formation conditions no matter how much pressure and heat are applied. For this reason, the supercritical fluid has unique properties, which are different from those of common liquid or gas. That is, when a supercritical state is reached, the density of the supercritical fluid becomes similar to that of a liquid phase, but the viscosity (molecular movement) becomes similar to a gaseous phase, and thus, the rates of diffusion and penetration into another materials are high due to the low viscosity (rapid molecular movement) similar to a gaseous phase, solvency is high due to the density similar to a liquid phase, and surface tension does not apply, and thus, it may easily penetrate even a fine area of other materials and shows a characteristic of high mass transfer rate.

A carbon dioxide phase changes depending on the conditions of temperature and pressure, and its effect on the foamed cell morphology inside a foam molded product is great. Since liquid carbon dioxide has stronger cohesiveness than kinetic power, the size of basic foamed cells is large, and it is generally difficult to control morphology of foamed nanocells by vaporization due to a rise in temperature. Further, since gaseous carbon dioxide has stronger kinetic power than cohesiveness, the size of basic foamed cells is large, and the kinetic power greatly reacts even to an insignificant temperature change, which has a fatal adverse effect on actual formation and adjustment of foamed microcells. However, since the cohesiveness and the kinetic power of the supercritical fluid at or above a critical point are in equilibrium even with changes in temperature and pressure, it is an optimal state for implementing foamed microcells. The thing to note here is that a pressure release rate and the curing time point of the thermocurable silicone resin should match after implementing foamed microcells using the supercritical fluid, and otherwise, uniform foamed microcells are not formed.

As a third step, further heating and pressurization are performed in the supercritical fluid state to form foamed microcells.

In an exemplary embodiment, a temperature condition for forming foamed microcells may be 31 to 60° C. and a pressure condition therefor may be 74 to 350 bar.

In an exemplary embodiment, the step of forming foamed microcells may be performed for 20 minutes to 12 hours.

In an exemplary embodiment, the third step may be divided into performing further first heating and pressurization in the supercritical fluid state by further pressurizing to target temperature and pressure at or above conditions where the supercritical fluid of carbon dioxide is formed, thereby forming a foaming nucleus; and performing second heating and pressurization to derive expansion by formation and diffusion of foam microcells in an area based on the foaming nucleus and saturate supercritical carbon dioxide for a certain period of time, thereby forming a final foamed microcellular structure.

In an exemplary embodiment, in the step of forming a foaming nucleus, the temperature condition for forming the foaming nucleus may be 31 to 45° C. and the pressure condition therefor may be 74 to 250 bar; and in the step of forming a foamed microcellular structure, the temperature condition for forming the foamed microcellular structure may be 45 to 60° C. and the pressure condition therefor may be 250 to 350 bar.

In an exemplary embodiment, the step of forming a foaming nucleus is performed for 10 minutes to 2 hours; and the step of forming a foamed microcellular structure may be performed for 10 minutes to 10 hours.

General supercritical fluid conditions of carbon dioxide are 31° C. and 74 bar, but carbon dioxide at or above a certain content should be added for a certain period of time for implementing actual uniform foamed microcells, and in this process, supercritical fluid formation conditions are not met, and heating and pressurization are performed stepwise in order to add the amount of the supercritical fluid stepwise.

A uniform foamed cellular structure is formed only by performing a saturation process in which carbon dioxide penetrates deep into the silicone resin by maintaining for a certain period of time in a state of being uniformly mixed without a boundary between a liquid phase and a gaseous phase.

In addition, it is preferred that the third step is performed for 8 hours or more. When it is performed for 7 hours or less, foamed cells may not be sufficiently formed, and when it is performed for 12 hours or more, the silicone resin may leak out of the mold.

As a fourth step, heating is performed to a temperature at which the thermocurable silicone resin is cured while pressurizing.

In an exemplary embodiment, a pressure reducing rate in the step of heating to the curing temperature may be 0.1 to 10 bar/s at a pressure at or above the conditions of the supercritical fluid state.

In an exemplary embodiment, a pressure reducing rate in the step of heating to the curing temperature may be 0.01 to 0.1 bar/s from the pressure of the supercritical fluid state condition or lower to 1 bar.

Most of the conventional injection and extrusion foaming methods have a mechanism of increasing a pressure reducing rate to increase a foaming ratio, but in the present invention, the size and the ratio of the foamed cells are determined in the pressurization and saturation processes, and in the depressurization process, the temperature is slowly raised simultaneously with pressure release to cure a silicone resin so that the foamed cells formed in the pressurization and saturation processes are not destroyed.

In order to maintain the foamed microcells formed in the previous step, the supercritical fluid inside the foamed cell should be slowly released and also the silicone resin should be cured so that the state is not deformed. Since the state of containing the supercritical fluid has very low thermal conductivity, the silicone resin is hardly cured under the temperature condition where the foamed microcells in the previous step are formed, and the curing starts only when the supercritical fluid is released to a certain level and the pressure is lowered to around 74 bar. An excessive pressure reducing rate may cause formation of large foamed cells, and in severe cases, even mold breakage.

In the depressurization, pressure release is performed to 0.1 to 10 bar/s at or above a pressure for forming the supercritical fluid so that the formed foamed microcells are not destroyed, and then at or below a pressure for forming the supercritical fluid, pressure release at 0.01 to 0.1 bar/s and heating to a temperature at which the curable silicone resin is cured may be performed stepwise.

In addition, in the depressurization process and the process of heating to the curing temperature, it is preferred for maintaining uniform foamed microcells that the temperature is not excessively raised during depressurization to 70 to 75 bar, and it is preferred, for forming small and uniform foamed cells, that the temperature is slowly raised to 60 to 85 while depressurizing to 70 to 0 bar.

Otherwise, the supercritical fluid gas which is trapped inside due to formation of an elastic body due to the cross-linked structure of the silicone resin and is not released causes expansion due to overfoaming by rapid vaporization due to a high temperature and subsequent rapid shrinkage. Further, the foaming ratio may be decreased due to the pressurizing effect by a rise in temperature.

In an exemplary embodiment, as an additional fifth step, the foamed microcellular structure may be stabilized by performing depressurization while maintaining it at or above the temperature at which the silicone resin is cured for a certain period of time.

In an exemplary embodiment, the stabilization step may be performed by depressurizing to 1 bar while maintaining 85 to 105° C.

In an exemplary embodiment, the stabilization step may be performed for 3 to 4 hours.

The supercritical fluid should be released as much as possible. Otherwise, the foamed cells may become large due to the presence of gaseous carbon dioxide.

In an exemplary embodiment, the pressure reducing rate in the stabilization step may be 0.001 to 0.01 bar/s at a pressure of 1 bar or less.

The depressurization may be performed as slow as possible, and when the pressure reducing rate is increased, carbon dioxide is replaced with high-temperature air to increase the size of foamed cells, and in severe cases, the silicone resin filled in the mold may swell to damage the mold.

In an exemplary embodiment, completing the final depressurization simultaneously with reaching the curing temperature, and then maintaining at the curing temperature for a certain period of time to stabilize the foamed microcellular structure may be included.

It is difficult to implement a uniform foamed cellular structure of 10 μm or less with a conventional technology using a thermoplastic due to the limitation of the material itself and the extrusion and injection manufacturing process.

However, the thermoplastic does not have a crystalline structure, has a small molecular weight, is a liquid or solid phase in a uniform state, and has a reactive group which causes a crosslinking reaction by heat at the end of the molecular structure.

In an exemplary embodiment, the silicone resin used in the present invention is a thermocurable type based on a platinum catalyst, and may be a liquid silicone rubber (LSR) type which is a liquid form at room temperature or a high consistency rubber (HCR) type which is a solid form at room temperature. The thermocurable silicone resin may be cured at 80 to 150° C.

Specifically, the HCR type silicone resin of the solid form is appropriate, and the silicone resin is a thermocurable type and may have a working time at room temperature of 1 to 5 hours, a curing time of about 10 minutes at 100 to 180° C., a post curing time of 2 to 4 hours at 150° C., a hardness (shore A) of 10 to 80, a tensile strength of 3 to 12 MPa, an elongation of 700 to 1,500%, a tear strength of 20 to 50 KN/m, and a specific gravity of 1 to 1.25 g/cm³.

More specifically, the thermocurable silicone resin of the solid form may have a working time at room temperature of 3 hours, a curing time of about 10 minutes at 100 to 120° C., a post curing time of 2 to 3 hours at 150° C., a hardness (shore A) of 10 to 30, a tensile strength of 4 to 10 MPa, an elongation of 1,200 to 1,500%, a tear strength of 24 to 32 KN/m, and a specific gravity of 1.00 to 1.10 g/cm³.

The characteristic value of the thermocurable silicone resin described above is a result value provided by measuring a specimen which is 100% completely cured by ASTM and ISO standards by the manufacturer. The present invention implements the foamed microcellular structure by using the thermocurable silicone resin manufactured and supplied by the manufacturer, and the foam molded product is 100% cured by an integrated batch foaming process and a sterilization process.

The carbon dioxide gas used in the present invention is a high-purity type for implementing a nano-level foamed microcellular structure.

In an exemplary embodiment, the carbon dioxide gas may have a purity of 99.99 to 99.999%.

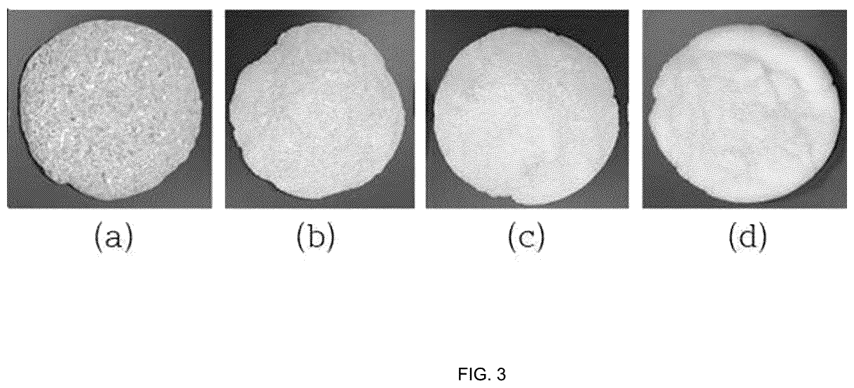

The appearance characteristics of the final molded article manufactured using ultra-high purity carbon dioxide gas of 99.99% or more show a significant difference from low-purity carbon dioxide gas and gases such as nitrogen. That is, the color of the final molded article is opaque, so that the foamed cells inside the molded article are invisible to the naked eye from the outside. The characteristics indirectly suggest that foamed microcells are formed inside (FIG. 3).

In an exemplary embodiment, the material of the mold used in the present invention may be a sus 304 or sus 316 material, and the size is determined depending on the size of the implant for insertion to a human body. A general size may be 200 to 800 cc, and specifically, may be 300 to 450 cc by volume. As the shape, a general shape may be a round and low or tall shape and an example of a special type may be a teardrop type and have a low or tall shape. Further, there may be various sizes and shapes according to the subject's needs. In addition, the mold used in the present invention may have fine holes in regular or irregular shapes and arrangements in the upper and lower portions of the mold. Herein, the size of the holes may be 0.1 to 1.2 mm. Further, the upper and lower portions of the mold may be fastened with a clamp and a similar shape and material including the clamp. The mold structure as such maintains input and discharge of ultra-high-purity carbon dioxide in a supercritical fluid state well, and minimizes the leakage of the supercritical fluid penetrated into the thermocurable silicone resin inside the mold to the outside in the process of expanding the thermocurable silicone resin with the supercritical fluid to maximize the dimensional stability and external aesthetics of the final molded article.

Meanwhile, the present invention provides a foamed microcellular silicone resin cured product manufactured by any one of the manufacturing methods.

In an exemplary embodiment, the morphology of the foamed microcellular silicone resin cured product to be implemented in the present invention may be characterized by a round, oval, or irregular closed cell structure of 1 to 10 μm.

Herein, the closed cell structure may be independent round or irregular closed cells of 1 to 3 μm. In addition, a structure in which 2 to 4 independent round or irregular closed cells of 1 to 3 μm are clumped together in the round, oval, or irregular closed cell structure of 10 μm or less may be included.

Herein, the size of the cell having excellent biocompatibility required in the present invention is 1 to 10 μm, and the width/length/thickness may be 10 μm or less. However, when two or more clumped cell lumps are continuously connected, the size of the connected site may be 10 μm or less (5 μm or less, if possible).

Since the foamed microcellular structure as such has a characteristic of allowing air to freely pass through without absorbing body fluids, it may be used as an implant for insertion to a human body, and it has a characteristic of having excellent biocompatibility with the body fluid of a human body.

In an exemplary embodiment, the cured product may be used as an implant which is inserted or attached to one or more selected from the group consisting of a chest, breasts, buttocks, a nose, a chin, a forehead, calves, thighs, and wrinkles of a human body.

The human body insertion type implant needs compatibility with a living body so that various types of symptoms are not experienced when it is inserted into a human body and present for a long time. For example, conventional silicone gel artificial breasts may cause a new type of cancer called anaplastic large cell lymphoma and various types of sequelae after transplant.

The present invention uses products which have passed biological stability testing as the silicone material itself and equipment with sterilization and extraction (for removing low molecular weights in the silicone material which may be possibly present) functions as supercritical fluid foaming equipment, and the final product has a small and fine foamed cellular structure to retain the characteristic of allowing oxygen to pass through and fluids or moisture not to pass when injected into the human body, and thus, has a very low possibility of occurring in vivo problems.

Hereinafter, the present invention will be described with reference to the examples of the present invention, but the examples are for easier understanding of the present invention, and the scope of the present invention is not limited thereby.

### Example 1

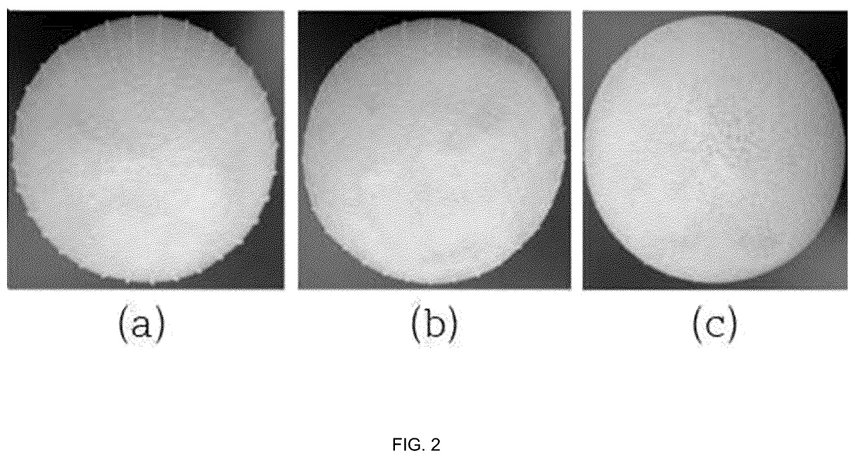

100 g of MED-4720 from Nusil which was a thermocurable silicone resin block type was taken and kneaded for about 10 minutes using kneader equipment. After completing kneading, 100 g of the kneaded product was added to a specially manufactured metal mold having a volume of 350 cc of high projection, the mold was fastened with a clamp and placed under the exact center of the high-temperature high-pressure container, and the container was covered. Ultra-high purity carbon dioxide having a purity of 99.999% was injected to the supercritical fluid formation conditions of 31° C. and 74 bar, and pressurization was performed to 250 bar while the temperature was gradually raised to 40° C. Then, additional pressurization was performed to 250 to 350 bar while the temperature was gradually raised to 50 to 55° C., and then saturation with the supercritical carbon dioxide was performed by maintaining for 5 hours. After completing saturation, curing was performed while depressurizing to 74 bar at 1 bar/s and to 1 bar at 0.01 bar/s, gradually heating to 85° C., and maintaining for 4 hours to stabilize the structure of foamed microcells, thereby manufacturing a final molded article (see (a) of FIG. 2).

### Example 2

100 g of MED-4720 from Nusil which was a thermocurable silicone resin block type was taken and kneaded for about 10 minutes using kneader equipment. After completing kneading, 100 g of the kneaded product was added to a specially manufactured metal mold having a volume of 350 cc of high projection, the mold was fastened with a clamp and placed under the exact center of the high-temperature high-pressure container, and the container was covered. Ultra-high purity carbon dioxide having a purity of 99.99% was injected to the supercritical fluid formation conditions of 31° C. and 74 bar, and pressurization was performed to 250 bar while the temperature was gradually raised to 40° C. Then, additional pressurization was performed to 250 to 350 bar while the temperature was gradually raised to 50 to 55° C., and then saturation with the supercritical carbon dioxide was performed by maintaining for 5 hours. After completing saturation, curing was performed while depressurizing to 74 bar at 1 bar/s and to 1 bar at 0.01 bar/s, gradually heating to 85° C., and maintaining for 4 hours to stabilize the structure of foamed microcells, thereby manufacturing a final molded article (see (b) of FIG. 2).

### Example 3

100 g of MED-4720 from Nusil which was a thermocurable silicone resin block type was taken and kneaded for about 10 minutes using kneader equipment. After completing kneading, 100 g of the kneaded product was added to a specially manufactured metal mold having a volume of 350 cc of high projection, the mold was fastened with a clamp and placed under the exact center of the high-temperature high-pressure container, and the container was covered. Carbon dioxide having a purity of 99.9% was injected to the supercritical fluid formation conditions of 31° C. and 74 bar, and pressurization was performed to 250 bar while the temperature was gradually raised to 40° C. Then, additional pressurization was performed to 250 to 350 bar while the temperature was gradually raised to 50 to 55° C., and then saturation with the supercritical carbon dioxide was performed by maintaining for 5 hours. After completing saturation, curing (crosslinking) was performed while depressurizing to 74 bar at 1 bar/s and to 1 bar at 0.01 bar/s, gradually heating to 85° C. which is the curing temperature condition of a silicone block, and maintaining for 4 hours to stabilize the structure of foamed microcells, thereby manufacturing a final molded article (see (c) of FIG. 2).

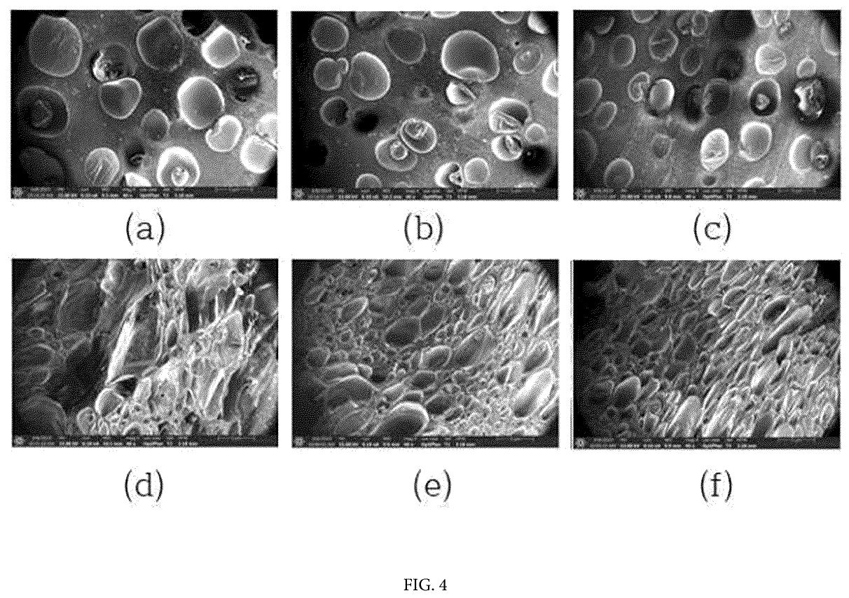

As confirmed in FIGS. 2 and 3, as the purity of carbon dioxide gas was higher, the color of the final molded article was closer to white, so that the foamed cells inside the molded article were invisible to the naked eye from the outside, and as confirmed in FIG. 4, the cell morphology was close to a small and uniform round shape. However, when the purity of carbon dioxide gas was low, the color of the final molded article became transparent, so that the foamed cells inside the molded article were visible to the naked eye from the outside, and the cells were shown to be large, uneven, and irregular morphology.

The morphology of the foamed cells using the supercritical carbon dioxide fluid reacts sensitively to a pressure release rate, time, and a rise rate to a curing temperature. As shown in FIG. 4, it was confirmed that when the saturation time was 30 minutes, the amount of the supercritical fluid penetrated into the silicone resin was small, and the pressure release rate and time and the curing temperature were not greatly affected, so that a cell shape close to a round shape was maintained, and when the saturation time was 5 hours, the amount of the supercritical fluid penetrated into the silicone resin was relatively large and the pressure release rate and time and the curing temperature were greatly affected, so that the cell shape was formed to be close to a long oval shape.

The method for manufacturing a foamed microcellular silicone resin cured product using a supercritical fluid according to the present invention may implement a silicone foam molded product which has uniform foaming and excellent dimensional accuracy and has a foamed cellular structure of 1 to 10 μm.

Since the foamed microcellular silicone resin cured product manufactured by the manufacturing method according to the present invention has uniform foaming and excellent dimensional accuracy and is 1 to 10 μm, it does not cause any problem of compatibility with a living body and may be applied to a human body insertion type implant.

The examples of the present invention described above and shown in the drawings should not be construed to limit the technical idea of the present invention. The protection scope of the present invention is limited only by the matters described in the claims, and a person with ordinary skill in the art to which the present invention pertains may improve and modify the technical idea of the present invention to various forms. Therefore, the improvement and modification belong to the protection scope of the present invention as long as they are evident to a person skilled in the art.

What is claimed is:

1. A method for manufacturing a foamed microcellular silicone resin cured product using a supercritical fluid, the method comprising:
(a) adding a thermocurable silicone resin to a container and injecting carbon dioxide;
(b) heating and pressurizing the carbon dioxide in the container to form a supercritical fluid state;
(c) further heating and pressurizing in the supercritical fluid state to form foamed microcells; and
(d) heating to a temperature at which the thermocurable silicone resin is cured while pressurizing.

2. The method of claim 1, wherein the step (c) includes, at or below the curing temperature of the step (d):
(c1) first heating and pressurizing in the supercritical fluid state to form a foaming nucleus; and
(c2) second heating and pressurizing to or above the temperature and the pressure of the step (c1) to form a foamed microcellular structure.

3. The method of claim 1, further comprising:
after the step (d), (e) performing depressurization while maintaining a temperature at or above the curing temperature of the step (d) for a certain period of time to stabilize the foamed microcellular structure.

4. The method of claim 1, wherein a temperature is from 31 to 60° C. and a pressure is from 74 to 350 bar for forming the foamed microcells in the step (c).

5. The method of claim 2, wherein:
in the step (c1), a temperature is from 31 to 45° C. and a pressure is from 74 to 250 bar for forming the foaming nucleus; and
in the step (c2), a temperature is from 45 to 60° C. and a pressure is from 250 to 350 bar for forming the foamed microcellular structure.

6. The method of claim 3, wherein the stabilizing of the foamed microcells of the step (e) is performed by depressurizing to 1 bar while maintaining a temperature from 85 to 105° C.

7. The method of claim 1, wherein a pressure reducing rate of the step (d) is from 0.1 to 10 bar/s at or above the pressure of a supercritical fluid state condition of the step (b).

8. The method of claim 1, wherein a pressure reducing rate of the step (d) is from 0.01 to 0.1 bar/s from the pressure of a supercritical fluid state condition or lower of the step (b) to 1 bar.

9. The method of claim 3, wherein a stabilization pressure reducing rate of the step (e) is from 0.001 to 0.01 bar/s at a pressure of 1 bar or less.

10. The method of claim 1, wherein the step (c) is performed for a time period from 20 minutes to 12 hours.

11. The method of claim 2, wherein:
the step (c1) is performed for a time period from 10 minutes to 2 hours; and
the step (c2) is performed for a time period from 10 minutes to 10 hours.

12. The method of claim 3, wherein step (e) is performed for a time period from 3 hours to 4 hours.

13. The method of claim 1, wherein the thermocurable silicone resin is cured at a temperature from 80 to 150° C.

14. The method of claim 1, wherein the carbon dioxide of step (a) has a purity from 99.9 to 99.999%.

15. A foamed microcellular silicone resin cured product manufactured by the method of claim 1, wherein the cured product has a round, oval, or irregular closed cell structure sized from 1 to 10 μm.

16. The foamed microcellular silicone resin cured product of claim 15, wherein the cured product is configured to be used as an implant which is inserted or attached to one or more parts of a human body selected from the group consisting of a chest, breasts, buttocks, a nose, a chin, a forehead, calves, thighs, and wrinkles.
